(19) United States
(12) Patent Application Publication
(10) Pub. No.: US 2025/0075164 A1
(43) Pub. Date: Mar. 6, 2025

(54) CELL CULTURE SYSTEM, CELL CULTURE APPARATUS, AND CELL IMAGE PICKUP SYSTEM

(71) Applicants: CANON KABUSHIKI KAISHA, Tokyo (JP); CANON MEDICAL SYSTEMS CORPORATION, Tochigi (JP)

(72) Inventors: HISAFUMI EBISAWA, Tokyo (JP); YOJI YAMAMOTO, Tokyo (JP); ICHIRO HARADA, Kanagawa (JP)

(21) Appl. No.: 18/816,061

(22) Filed: Aug. 27, 2024

(30) Foreign Application Priority Data
Aug. 30, 2023 (JP) 2023-139700

(51) Int. Cl. C12M 1/34; C12M 1/00

(52) U.S. Cl. CPC C12M 41/36; C12M 23/22; C12M 29/00; C12M 31/02

(57) ABSTRACT

A cell culture system includes: a cell culture apparatus including: a placement table placing a culture vessel having a culture surface on which cell culture is viable and a side surface forming a peripheral wall around the culture surface, the side surface being optically transparent; a supply pipe allowing liquid to be supplied into the culture vessel; and a discharge pipe allowing liquid to be discharged from the culture vessel; an image pickup portion allowing observation of the culture surface; and a light irradiation portion emitting illumination light toward the side surface, wherein the supply pipe and the discharge pipe are arranged so that an openings thereof are located in proximity to points on an inner side surface of the culture vessel, which define a width of the culture surface in a direction orthogonal to an optical axis of the illumination light.

### BACKGROUND OF THE INVENTION

### Field of the Invention

The present disclosure relates to a cell culture system, a cell culture apparatus, and a cell image pickup system.

### Description of the Related Art

In cell culture, a technology of enabling observation of a large area of a culture surface in an automated manner is important for achievement of automated cell culture. Further, when a colony, which is a cluster of cells, is formed at the time of observation of the culture surface, a shape of the colony of cultured cells can be observed based on scattering of irradiated light. In Japanese Patent Application Laid-Open No. 2016-077226 (Patent Literature 1), there is proposed a system that enables observation of a shape of a colony by radiating light through a side surface of a culture vessel and observing scattered light from the colony with a camera installed below the culture vessel.

An automated cell culture apparatus requires supply and discharge of water to and from a culture medium. Thus, pipes such as tubes are installed around the culture vessel. However, the system disclosed in Japanese Patent Application Laid-Open No. 2016-077226 is not configured in consideration of the pipes described above.

### SUMMARY OF THE INVENTION

The present disclosure has been made in view of the circumstances described above. More specifically, one object of the present disclosure is to provide a cell culture apparatus, a cell culture system, and a cell image pickup system, which enable automated cell culture using pipes for supplying and discharging water into and from a culture vessel.

In order to solve the above-mentioned problem, according to an aspect of the present disclosure, there is provided a cell culture system including:
a cell culture apparatus including:
   a placement table configured to allow placement of a culture vessel having a culture surface on which cell culture is viable and a side surface that forms a peripheral wall around the culture surface, the side surface being optically transparent;
   a supply pipe configured to allow liquid to be supplied into the culture vessel; and
   a discharge pipe configured to allow liquid to be discharged from the culture vessel;
an image pickup portion configured to allow observation of the culture surface; and
a light irradiation portion configured to emit illumination light toward the side surface,
wherein the supply pipe and the discharge pipe are arranged so that an opening of the supply pipe and an opening of the discharge pipe are located in proximity to points on an inner side of the culture vessel, which define a width of the culture surface in a direction orthogonal to an optical axis of the illumination light.

Further features of the present invention will become apparent from the following description of exemplary embodiments with reference to the attached drawings.

### BRIEF DESCRIPTION OF THE DRAWINGS

FIG. 1A is a view for illustrating a schematic configuration of a cell culture system according to a first embodiment of the present disclosure.

FIG. 1B is a view for illustrating the schematic configuration of the cell culture system illustrated in FIG. 1A when viewed from above.

FIG. 2A is a view for illustrating a positional relationship between a light irradiation portion and a culture vessel according to the first embodiment.

FIG. 2B is a view for illustrating a schematic configuration of the culture vessel illustrated in FIG. 2A and pipes when viewed from above.

FIG. 2C is a view for illustrating a schematic configuration of the culture vessel illustrated in FIG. 2A and the pipes in different arrangements with respect to the culture vessel when viewed from above.

FIG. 3A is a view for illustrating a positional relationship between the light irradiation portion and the culture vessel according to the first embodiment.

FIG. 3B is a view for illustrating a schematic configuration of the culture vessel illustrated in FIG. 3A and the pipes when viewed from above.

FIG. 3C is a view for illustrating a schematic configuration of the culture vessel illustrated in FIG. 3A and the pipes in a different arrangement with respect to the culture vessel when viewed from above.

FIG. 4A is a view for illustrating one modification example of light shielding for a bottom portion of the vessel according to the first embodiment.

FIG. 4B is a view for illustrating another modification example of the light shielding for the bottom portion of the vessel according to the first embodiment.

FIG. 5 is a view for illustrating a positional relationship among a light irradiation portion, a culture vessel, and pipes according to a second embodiment.

FIG. 6 is a view for illustrating a positional relationship among a light irradiation portion, a culture vessel, and pipes according to a third embodiment.

FIG. 7A is a view for illustrating a schematic configuration of a cell culture system according to a fourth embodiment.

FIG. 7B is a view for illustrating a state in which illumination light is being radiated in the cell culture system illustrated in FIG. 7A.

### DESCRIPTION OF THE EMBODIMENTS

Hereinafter, exemplary embodiments of the present disclosure are described in detail with reference to the accompanying drawings. However, for example, dimensions, materials, shapes, and relative positions of components described in the following embodiments may be freely selected except for specific conditions described below, and can be changed depending on a configuration of an apparatus to which the present disclosure is applied or on various conditions. Further, components having substantially the same or functionally similar configurations in this specification and the drawings are denoted by the same reference symbols throughout the drawings, and an overlapping description thereof is omitted.

In cell culture, a percentage of cells to a culture surface (cell confluence) serves as an index of timing of passage. Thus, it is desirable to pick up an image of cells, which are phase objects distributed over a large area, within a short period of time. In order to observe a large area within a short period of time, a low-power lens is generally used. However, the use of a low-power lens is not suitable for a phase-contrast microscope using transmitted light. Thus, in photographing cells with a low-power lens over a large area, it is preferred that light be radiated through a side surface of a culture vessel and an image of scattered light from cells be picked up from a bottom surface side.

Further, when automated cell culture is carried out, as described above, pipes for supplying and discharging, for example, water to and from a culture medium are often provided to each culture vessel (for example, a petri dish). It is considered that, when parallel light which can illuminate a large area is radiated to a culture medium through a side surface of a petri dish so as to pick up an image of cells, a part of radiated light may reach the pipes and be reflected by the pipes.

Thus, when light is radiated through the side surface of the culture vessel so as to pick up an image of cells, it is considered that light reflected from the pipes (hereinafter referred to as “stray light”) is required to be taken into consideration. Further, when parallel light is intended to be radiated through the side surface of the culture vessel to a large area of the culture vessel, it is required that not only stray light from the pipes, which has been exemplified above, but also stray light generated due to, for example, a shape of the culture vessel (for example, a protrusion arranged on a bottom surface) be taken into consideration. As described above, in a cell culture apparatus including, for example, pipes, it is considered that a more suitable cell image can be obtained by reducing the stray light generated due to the pipes or reducing an area that the stray light can reach. In order to cope with the problem occurring due to the presence of the pipes, a configuration that enables a reduction in the stray light or a reduction in the area that may be affected by the stray light is described in the following embodiments.

### First Embodiment

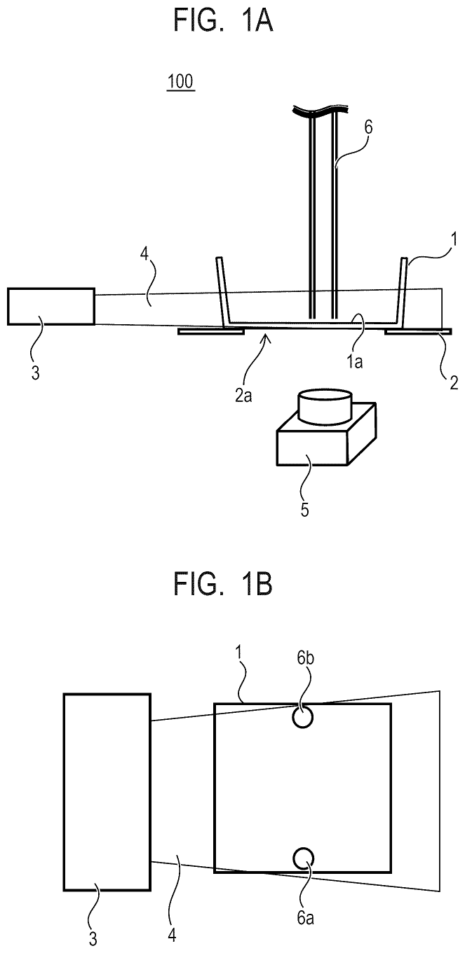

Now, a cell culture system according to a first embodiment of the present disclosure is described with reference to FIG. 1A through FIG. 4B. FIG. 1A is a view for illustrating a cell culture system 100 according to this embodiment, and FIG. 1B is a plan view of the configuration illustrated in FIG. 1A when viewed from above (schematic configuration in plan view). For convenience of the description, a culture vessel is held in a horizontal plane, and top, bottom, right, left, and lateral sides are defined based on the horizontal plane and a light irradiation direction in the following description. However, these definitions are not intended to limit the contents of the disclosure. The above-mentioned directions can be appropriately changed in accordance with, for example, a shape of a culture vessel, a light irradiation direction, and circumstances under which the cell culture system is installed. Further, directions used for the description are not exactly precise and may include manufacturing errors, for example, an error occurring when a culture vessel is placed on a placement table and an error in parallelism associated with parallelism of illumination light, and errors being allowable at a time when the disclosure is carried out.

### (Configuration of Cell Culture System)

As illustrated in the schematic view of FIG. 1A, the cell culture system 100 according to this embodiment includes a placement table 2, a light source 3, an image pickup apparatus 5, and pipes 6. A culture vessel 1 in which cells are to be cultured is placed on the placement table 2. The culture vessel 1 is a storage vessel that stores a culture medium for culturing cells. A culture surface 1a that allows adhesion and proliferation of cells is provided on a bottom portion of the vessel. In the culture vessel 1, the culture surface 1a may be a bottom surface of the vessel or a component provided on the bottom surface of the vessel, which is different from the vessel.

The placement table 2 has a hole 2a corresponding to an image pickup area. The hole 2a allows the image pickup apparatus 5 arranged below the placement table 2 to pick up an image of cells in the culture vessel 1 through a transparent bottom surface of the culture vessel 1. The light source 3 emits illumination light 4, which is to be radiated to cells through a side surface of the culture vessel 1, from a side of the culture vessel 1. The image pickup apparatus 5, which is located below the culture vessel 1, picks up an image of scattered light, which results from scattering of light radiated to cells adhering to the culture surface 1a, from below to obtain a shape of a colony. The pipes 6 include a supply pipe 6a and a discharge pipe 6b. The supply pipe 6a allows water and a sample such as a culture medium to be supplied into the culture vessel 1. The discharge pipe 6b allows water to be discharged from the culture vessel 1.

### (Culture Vessel)

As described above, the culture vessel 1 stores a culture medium for culturing cells and cells contained in the culture medium. A transparent resin and glass, which are easy to process and allow easy observation, are suitable as a material of the culture vessel 1. When a resin is used, polystyrene, polycarbonate, or acrylic can be used. Further, a culture vessel with a bottom surface having a rectangular or circular shape is exemplified as a shape of the culture vessel 1. The culture surface 1a is formed so as to allow adhesion and proliferation of cells. A transparent material that allows easy observation is preferred as a material of the culture surface 1a. In view of costs and production, it is preferred that a part of the culture surface 1a be made of the same material as that of the culture vessel 1. In this case, it is preferred that a surface treatment such as a plasma treatment be performed on the culture surface 1a so that cells are more likely to adhere only to the culture surface 1a.

### (Light Irradiation Portion)

It is preferred that the light source 3 be able to emit light having a wavelength that allows the image pickup apparatus 5 to acquire an image without adversely affecting cells as the illumination light 4. A halogen lamp or an LED light source can be used as the light source 3. The light source 3 according to this embodiment is exemplified as one example of the light irradiation portion in the present disclosure, which includes, for example, a collimating unit described later, depending on conditions. In view of contrast of image quality, it is preferred that stray light be reduced as much as possible in order to acquire only scattered light from the cells. Accordingly, it is preferred that the illumination light 4 be radiated only to the cells on the culture surface 1a. Further, it is preferred that a pickup image surface be without unevenness. Thus, it is preferred that the illumination light 4 have substantially uniform intensity and wavelength range in an image pickup area. As described above, a light source that radiates a uniform amount of light only to the culture surface is preferred to be selected. For example, it is preferred that a line-type LED light source having high directivity or a line light guide including bundled fibers having output ends being linearly arranged be used.

The use of the light source 3 described above enables efficient illumination of only the culture surface 1a and the vicinity thereof. The illumination light 4 emitted from the light source 3 is radiated through the side surface of the culture vessel 1. Thus, the illumination light 4 has a difference in illumination intensity between an incident side and an exit side of the culture vessel 1a. In order to reduce the difference, it is preferred that illumination light with a reduced spread angle, which is closer to parallel light, be used. Thus, it is effective to use a collimating unit for the light source 3 so as to obtain parallel light as the illumination light 4. As the collimating unit, for example, a cylindrical lens is exemplified. Further, providing a plurality of light sources to achieve multi-directional illumination is also effective to obtain more uniform illumination light.

Further, as a method of limiting an illumination area by the light source 3 to the culture surface 1a, it is also effective to provide a mask on the side surface of the culture vessel 1. The mask can be provided between the light source 3 and the culture surface 1a. When the mask is provided, it is less necessary to use line-type light as the illumination light 4 to be emitted from the light source 3. Thus, when the mask is used, a light source for emitting light that can illuminate a large area can be selected although efficiency is lowered.

### (Image Pickup Apparatus)

The image pickup apparatus 5 is used to acquire an image of cells adhering to the culture surface 1a. For example, a so-called digital camera using a CMOS and a CCD can be used as the image pickup apparatus 5.

### (Pipes)

The pipes 6 are used to supply or discharge a test solution for a culture medium into or from the culture vessel 1. In this embodiment, a pair of pipes 6, that is, a supply pipe 6a that is used to supply a test solution or water and a discharge pipe 6b that is used to discharge the test solution or water are provided to one culture vessel 1. However, the number of pipes is not limited to two, and a larger number of pipes may be provided to one culture vessel. The pipes 6 can be used to, for example, supply and discharge water to and from a culture medium at the time of replacement of the culture medium. When the illumination light 4 is incident on the pipes 6, reflected light from the pipes 6 turns into stray light. The stray light may be a factor in reduction of quality of a cell image picked up by the image pickup apparatus 5. Thus, it is preferred that a material having a low reflectance be used for the pipes 6 in order to reduce the stray light described above. It is preferred that a transparent resin (for example, silicone or polystyrene) be used.

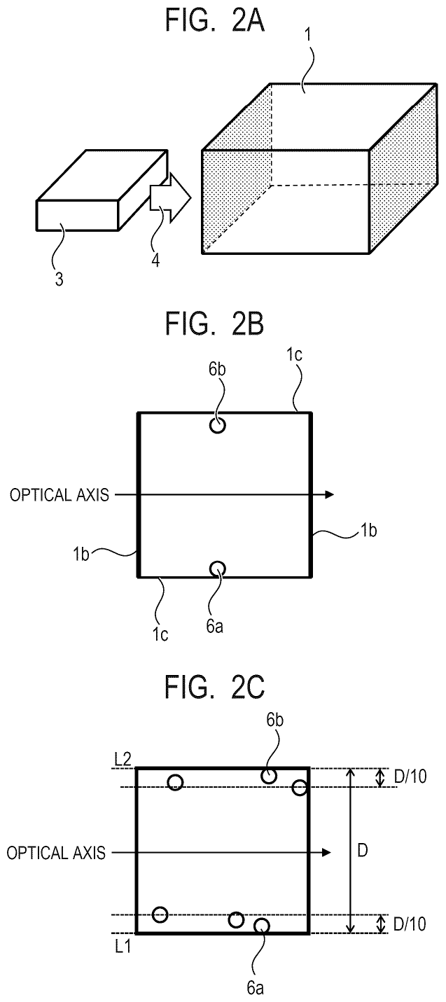

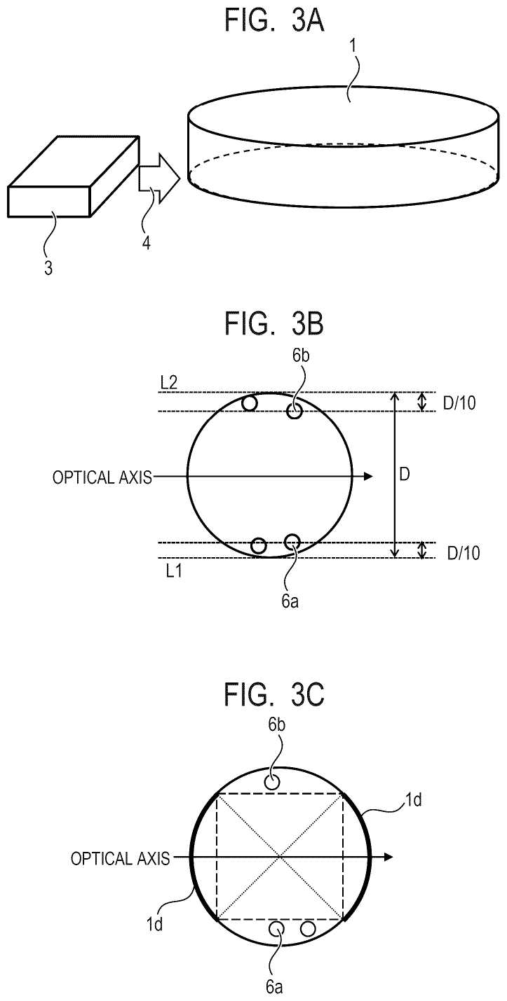

However, even when a material having a reflectance as low as possible is selected, intensity of the reflected light is often higher than intensity of the scattered light from the cells. Thus, it is preferred that positions of the pipes with respect to the illumination light 4 be determined so that the illumination light having high intensity be less liable to be incident on the pipes 6 or the reflected light from the pipes 6 be less liable to affect the image pickup area. The arrangements of the pipes 6 in view of the above-mentioned problems are described below with reference to FIG. 2A to FIG. 3C.

FIG. 2A to FIG. 2C are views for illustrating a positional relationship between the light source 3 and the culture vessel 1 and a positional relationship among the culture vessel 1 and the pipes 6 provided to the culture vessel 1. A positional relationship between the light source 3 and the culture vessel 1 having a rectangular bottom surface (having a rectangular parallelepiped box-like shape) is schematically illustrated in FIG. 2A. An example of a positional relationship among side surfaces of the culture vessel 1 and the pipes 6 is schematically illustrated in FIG. 2B. Further, another example of the positional relationship among the side surfaces of the culture vessel 1 and the pipes 6 is illustrated in FIG. 2C.

As illustrated in FIG. 2B, two pipes 6a and 6b are arranged in proximity not to side surfaces 1b, which are side surfaces on an incident side and an exit side for the illumination light 4, but to side surfaces 1c other than the side surfaces 1b, which are parallel to an optical axis of the illumination light 4, respectively. The above-mentioned arrangement of the pipes 6a and 6b allows an area that may be affected by the stray light to be positioned outside the image pickup area and as far as possible from the image pickup area and thus enables a more suitable cell image of a larger area to be obtained.

In order to reduce an influence of the stray light as much as possible, it is desirable that the pipes 6 be arranged in contact with the side surfaces 1c being parallel to each other. In consideration of a process of bringing the pipes 6 closer to the culture vessel 1, however, it is not practical to actually arrange the pipes 6 in contact with the culture vessel 1. Thus, the pipes 6 are arranged in proximity to the side surfaces 1c being parallel to each other. Here, the term “proximity” defines a state in which the pipes 6 are arranged in the vicinity of the side surfaces 1c without causing a problem when the above-mentioned process is carried out.

Next, a specific arrangement of the pipes 6, which enables the influence on the stray light to be reduced to such a level that the colony can be observed, is described with reference to FIG. 2C. Here, a width of the culture surface 1a of the culture vessel 1 in a direction perpendicular to the optical axis is represented by D, and straight lines extending in an extending direction of inner surfaces of the culture vessel 1, which are parallel to the optical axis, are represented by L1 and L2, respectively. At this time, the pipes 6 are arranged at positions in proximity to the inner surfaces of the culture vessel 1, which define the width D. More specifically, the pipes 6 are arranged so as to in a plane view, make a distance between a foot of a perpendicular extended from a center of an opening of the pipe 6a to the culture surface 1a and one of the straight lines L1 and L2, which is closer to the pipe 6a, equal to D/10 or less and, similarly, to make a distance between a foot of a perpendicular extended from a center of an opening of the pipe 6b to the culture surface 1a and one of the straight lines L1 and L2, which is closer to the pipe 6b, equal to D/10 or less. When the pipes 6 are arranged so as to satisfy the above-mentioned conditions, the pipes 6 can easily be brought closer to the culture vessel 1 and a suitable cell image of a larger area can be obtained.

Next, the arrangement of the pipes 6 when the culture vessel 1 has a circular bottom surface (a cylindrical shape with a circular cross section) is described with reference to FIG. 3A to FIG. 3C. A positional relationship between the light source 3 and the culture vessel 1 in the above-mentioned case is schematically illustrated in FIG. 3A, a positional relationship among a side surface of the culture vessel 1 and the pipes 6 are schematically illustrated in FIG. 3B and FIG. 3C.

When the culture vessel 1 has a circular bottom surface, a diameter of the culture surface 1a in a direction perpendicular to the optical axis is represented by D, and straight lines extending in an extending direction of tangents to an inner surface of the culture vessel 1, which are parallel to the optical axis, are represented by L1 and L2, respectively. At this time, the pipes 6 are arranged at positions in proximity to the inner surface of the culture vessel 1, which define the width D. More specifically, the pipe 6a is arranged so as to in a plane view, make a distance between a foot of a perpendicular extended from a center of an opening of the pipe 6a to the culture surface 1a and one of the straight lines L1 and L2, which is closer to the pipe 6a, equal to D/10 or less, and similarly, the pipe 6b is arranged so as to in a plane view, make a distance between a foot of a perpendicular extended from a center of an opening of the pipe 6b to the culture surface 1a and one of the straight lines L1 and L2, which is closer to the pipe 6b, equal to D/10 or less. When the pipes 6 are arranged so as to satisfy the above-mentioned conditions, even in the culture vessel 1 having a circular bottom surface, the pipes 6 can easily be brought closer to the culture vessel 1 and a suitable cell image of a larger area can be obtained.

Also when the pipes 6 are arranged as follows, the influence of the stray light can be reduced to such a level that the colony can be observed. More specifically, when the culture vessel 1 has a circular bottom surface, regions between the circle and sides of a square inscribed in the circle in plan view, the sides extending perpendicularly to the optical axis, are defined as regions 1d in which the pipes 6 are not allowed to be arranged. In this case, when the pipes 6 are arranged between the sides extending in parallel to the optical axis and the inner surface of the culture vessel 1, an area that may be affected by the stray light can be positioned outside the image pickup area and as far as possible from the image pickup area. Thus, a more suitable cell image of a larger area can be obtained.

In the first embodiment, the cell culture system 100 in which cell culture is carried out while the light source 3, the placement table 2, the image pickup apparatus 5, and the pipes 6 are fixed has been described. However, a configuration to which the present disclosure is applicable is not limited to the cell culture system described above. For example, the placement table 2 and the pipes 6, which correspond to one culture vessel 1, are provided to the culture vessel 1. The pipes 6 and the culture vessel 1 are arranged so as to satisfy the positional relationship described above, and a cell culture apparatus having a plurality of configurations described above may also be formed. An image pickup system including the light source 3 and the image pickup apparatus 5 corresponding thereto can be added to the cell culture apparatus described above. The light source 3 is configured to emit the illumination light 4 so as to satisfy the above-mentioned relationship with respect to the pipes 6. In this case, it is desirable that the image pickup system and the cell culture apparatus be able to change their positions relative to each other so that the image pickup system can pick up an image of, for example, cells in a plurality of culture vessels. When the image pickup system described above is additionally provided to the cell culture apparatus, a cell image pickup system according to the present disclosure can be constructed.

As described above, the cell culture system 100 according to the present disclosure includes the cell culture apparatus, the image pickup portion (image pickup apparatus 5), and the light irradiation portion (light source 3). In this case, the cell culture apparatus includes the placement table 2 and the pipes 6 including the supply pipe 6a and the discharge pipe 6a. The culture vessel 1 is placed on the placement table 2. The culture vessel 1 has the culture surface 1a on which cell culture is viable and the side surface that forms a peripheral wall around the culture surface 1a. The side surface is optically transparent. The supply pipe 6a is used when liquid is supplied into the culture vessel, and the discharge pipe 6b is used when liquid is discharged from the culture vessel. The image pickup apparatus 5 is used for observation of the culture surface 1a. The illumination light 4, which is radiated toward the side surface of the culture vessel 1, is emitted from the light source 3. In the cell culture system 100 according to the first embodiment, the supply pipe 6a and the discharge pipe 6b are arranged so that the opening of the supply pipe 6a and the opening of the discharge pipe 6b are located in proximity to points on the inner side surface of the culture vessel 1, which define the width D of the culture surface 1a in the direction orthogonal to the optical axis of the illumination light 4.

When an image of cells is to be obtained, it is preferred that the following conditions be satisfied as conditions for reducing the influence of the stray light from the pipes on the image to a suitable level. The width of the culture surface 1a is represented by D, and two straight lines that pass through points on the inner side surface of the culture vessel 1 and are parallel to the optical axis are represented by L1 and L2. In this case, the supply pipe 6a and the discharge pipe 6b are preferred to be arranged so that a distance between the foot of the perpendicular extended from the center of the opening of one of the pipes 6a and 6b to the culture surface 1a and one of the straight lines L1 and L2, which is closer to the corresponding pipe, becomes equal to D/10 or less and the foot of the perpendicular extended from the center of the opening of another one of the pipes 6a and 6b to the culture surface 1a and one of the straight lines L1 and L2, which is closer to the corresponding pipe, becomes equal to D/10 or less.

Further, in the cell culture system 100, the culture vessel 1 may have a rectangular shape (a rectangular bottom surface, that is, a rectangular culture surface) in plan view. In such a case, the supply pipe 6a and the discharge pipe 6b are preferred to be arranged in proximity to the inner side of the side surfaces 1c of the culture vessel 1, the side surfaces 1c extending in parallel to the optical axis. Further, the culture vessel 1 may have a circular shape (a circular bottom surface, that is, a circular culture surface) in plan view. In such a case, the supply pipe 6a and the discharge pipe 6b are preferred to be arranged between opposed surfaces of a square frame inscribed in the circle in plan view, the opposed surfaces being parallel to the optical axis, and a surface defining the inner side of the circle opposed to the opposed surfaces. The culture vessel 1 having a rectangular or circular shape includes two regions that satisfy the above-mentioned conditions. When at least one of the supply pipe 6a or the discharge pipe 6b is arranged in any one of the regions, another one of the pipes can be arranged in another one of the regions. More specifically, both of the supply pipe 6a and the discharge pipe 6b can be arranged in one of the two regions that satisfy the conditions described above. Alternatively, one of the supply pipe 6a and the discharge pipe 6b may be arranged in any one of the above-mentioned regions, and another one of the supply pipe 6a and the discharge pipe 6b may be arranged in another one of the above-mentioned regions.

In the cell culture system 100 described above, it is desirable that only the cells be irradiated with the illumination light 4 in order to, for example, prevent the generation of stray light due to irradiation of regions of the culture vessel 1 other than a desired region. Thus, the illumination light 4 is preferred to be sheet-like parallel light. Further, the present disclosure can also be applied to the cell culture apparatus including the placement table 2, the supply pipe 6a, and the discharge pipe 6b, which are described above. In this case, the present disclosure can be applied to a cell image pickup system including the cell culture apparatus, the light irradiation portion (light source 3), and the image pickup portion (image pickup apparatus 5). In this case, the supply pipe 6a and the discharge pipe 6b are preferred to be arranged so that their openings are located in proximity to the points on the inner side surface of the culture vessel 1, which define the width of the culture surface 1a in the direction orthogonal to a center axis of the culture vessel 1 placed on the placement table 2 in plan view. The light irradiation portion is only required to be moved relative to the cell culture apparatus and be able to irradiate the side surface of the culture vessel 1 with the illumination light so that the optical axis matches with the center axis described above. The image pickup apparatus 5 is only required to be able to pick up an image for observation of the culture surface illuminated with the illumination light in response to the illumination light.

### Modification Examples of First Embodiment

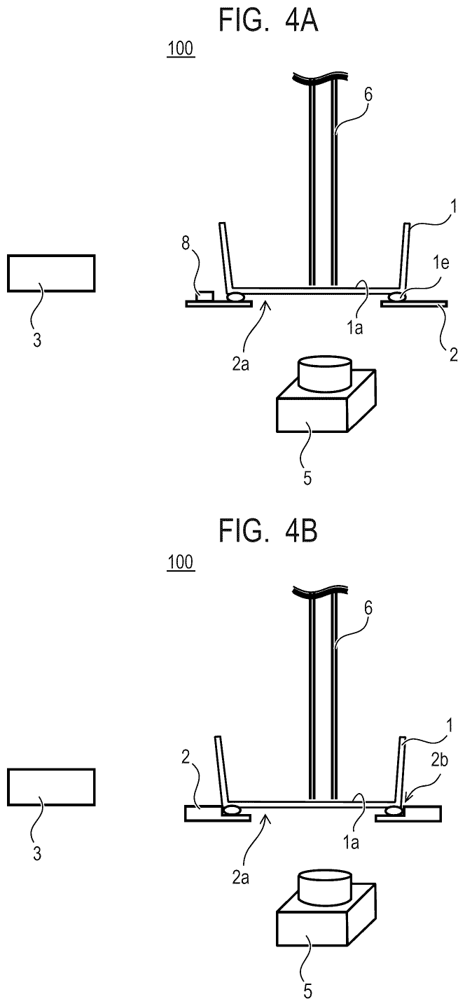

It is conceivable to use, for example, a commercially available petri dish as the culture vessel 1. A commercially available petri dish often has a protrusion on a peripheral portion of its lower side so as to prevent contact of a bottom surface of the petri dish with a ground surface. When the illumination light is incident on the protrusion, the light spreads in multiple directions, which may generate stray light. The modification examples have an object to cope with a problem occurring when a petri dish having a protrusion as described above is used. Configurations included in the modification examples are described with reference to FIG. 4A and FIG. 4B. FIG. 4A and FIG. 4B correspond to FIG. 1A of the first embodiment. Thus, the same configurations are denoted by the same reference symbols, and a description thereof is omitted here.

In a first modification example illustrated in FIG. 4A, a culture vessel 1 has a protrusion 1e on its bottom surface. When illumination light 4 emitted from a light source 3 is incident on the protrusion 1e, the stray light described above is generated. In this modification example, a mask 8 is provided between the protrusion 1e and the light source 3 so as to shield the protrusion 1e from irradiation with the illumination light 4. In this manner, the protrusion 1e is not irradiated with the illumination light 4, and thus the stray light, which is generated due to the protrusion 1e, can be prevented from being picked up as an image by the image pickup system 5.

Further, a method of shielding the protrusion 1e from the irradiation with the illumination light is not limited to the use of the mask 8. For example, a configuration of setting the protrusion 1e outside an irradiation area with the illumination light on a placement table 2 may be added. Such a configuration is described as a second modification example with reference to FIG. 4B. In a configuration illustrated in FIG. 4B, a placement table 2 has a recess 2b having a depth corresponding to a thickness of a protrusion 1e (distance from a bottom portion to a top portion of the protrusion in a direction perpendicular to a bottom surface of a culture vessel 1). When the culture vessel 1 is placed on the placement table 2 described above, the protrusion 1e is not irradiated with illumination light and only a culture surface 1a is irradiated with the illumination light emitted from the side. The recess that is formed so as to receive the entire peripheral area of the bottom portion of the culture vessel 1 is exemplified. However, a recess may be formed so as to receive only the protrusion 1e. A shape of the recess is not limited to the example described here as long as the irradiation with the illumination light can be avoided.

When a configuration of shielding the protrusion formed on the bottom surface of the culture vessel 1 from the irradiation with the illumination light is provided to the cell culture apparatus according to the first embodiment as described above, stray light generated due to the protrusion can be reduced. Thus, a more suitable cell image of a larger area can be obtained.

As described above, the cell culture system 100 according to the present disclosure includes the cell culture apparatus, the image pickup portion (image pickup apparatus 5), and the light irradiation portion (light source 3). In this cell culture system, the cell culture apparatus includes the placement table 2 and the pipes 6 including the supply pipe 6a and the discharge pipe 6b. The culture vessel 1 is placed on the placement table 2. The culture vessel 1 has the culture surface 1a on which cell culture is viable and the side surface that forms the peripheral wall around the culture surface 1a. The side surface is optically transparent. The supply pipe 6a is used when liquid is supplied into the culture vessel, and the discharge pipe 6b is used when liquid is discharged from the culture vessel. The image pickup apparatus 5 is used for the observation of the culture surface 1a. The illumination light 4, which is radiated toward the side surface of the culture vessel 1, is emitted from the light source 3. In the cell culture system according to the modification examples, the culture vessel 1 has the protrusion 1e that is formed on a surface opposite to the culture surface 1a and protrudes in a direction opposite to a direction toward the culture surface 1a. The placement table 2 can have a configuration of shielding the protrusion 1e from the illumination light 4. As the configuration, for example, the mask 8 or the recess 2b can be used. The mask 8 is provided between the light source 3 and the protrusion 1e. The recess 2b is formed in a surface of the placement table 2 and receives the protrusion 1e when the culture vessel 1 is placed on the placement table 2.

### Second Embodiment

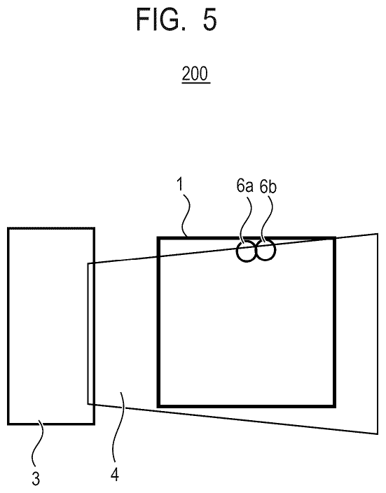

In the first embodiment described above, the supply pipe 6a that is used when, for example, water is supplied to the culture vessel and the discharge pipe 6b that is used when water is discharged therefrom are arranged so as to be separated apart from each other. Meanwhile, in this embodiment, a supply pipe 6a and a discharge pipe 6b are arranged together as one set. A cell culture system 200 having the configuration described above according to this embodiment is described with reference to FIG. 5. In FIG. 5, a main configuration is illustrated in the same fashion as in FIG. 1B.

As illustrated in FIG. 5, in this embodiment, the supply pipe 6a and the discharge pipe 6b are provided so as to be in contact with each other. The supply pipe 6a and the discharge pipe 6b are integrally brought closer to the culture vessel 1 as a set of the pipes 6. In this embodiment, when the conditions for the arrangement of one of the pipes 6a and 6b described in the first embodiment are applied to the pipes 6 as a set of the pipes 6 that are provided integrally, the same effects as those obtained in the first embodiment are obtained. When two pipes are integrally provided as one set, the number of sources of generation of the stray light can be reduced to one. Thus, the effects of further reducing the influence of the stray light can be obtained as compared to the first embodiment. In FIG. 5, the culture vessel 1 having a rectangular bottom surface is exemplarily illustrated. The configurations or the conditions, which have been described with reference to FIG. 3A to FIG. 3C, are also applicable to a case in which the culture vessel 1 has a circular bottom surface as exemplarily illustrated in FIG. 3A to FIG. 3C.

### Third Embodiment

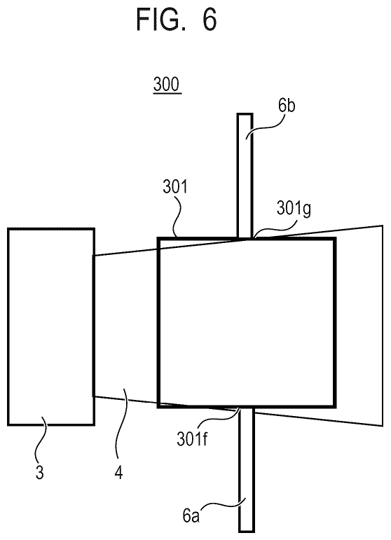

The configurations that reduce stray light at the time of picking up an image of cultured cells, which are achieved by arranging the pipes 6 with respect to the culture vessel 1 so that, for example, water is supplied or discharged through the opening located above the culture vessel 1, have been described in the first and second embodiments. In the above-mentioned configurations, a commercially available culture vessel can generally be used as the culture vessel 1. Meanwhile, it is also conceivable to process a culture vessel so as to reduce stray light. In this embodiment, two holes (openings) for supplying and discharging water are formed in a side surface of a culture vessel, and pipes are connected to the openings, respectively. Specifically, pipes 6 are directly connected to the openings formed in the side surface of the culture vessel. A cell culture system 300 having the configuration described above according to this embodiment is described with reference to FIG. 6. In FIG. 6, a main configuration is illustrated in the same fashion as in FIG. 1B.

In this embodiment, a culture vessel 301 has a hole 301f corresponding to a supply pipe 6a and a hole 301g corresponding to a discharge pipe 6b. When the culture vessel 301 exemplarily illustrated in FIG. 6 has a rectangular bottom surface, the holes 301f and 301g are formed in side surfaces extending in parallel to a direction of an optical axis of illumination light 4. The above-mentioned arrangement of the holes and connection of pipes through the holes can substantially prevent the pipes 6 from being present within an irradiation area with the illumination light 4. Specifically, in this embodiment, the pipes 6 are prevented from being arranged within the irradiation area with the illumination light 4 at the time of picking up an image of cultured cells. Thus, stray light generated due to the illumination light 4 being incident on the pipes 6 can be reduced.

The culture vessel 301 having a rectangular bottom surface is described with reference to FIG. 6. When the culture vessel 301 has a circular bottom surface, holes 301a and 301b are preferred to be formed, for example, on an inner surface of the culture vessel 301, which defines the width D in FIG. 3B. Alternatively, the holes 301a and 301b are preferred to be formed in the inner surface of the culture vessel 301 so as to fall within a range of D/10 in a direction of the width D from the inner surface of the culture vessel 301, which defines the width D. As a result, the stray light generated due to the illumination light 4 being incident on the pipes 6 can be reduced. The connection of both of the supply pipe 6a and the discharge pipe 6b through the holes formed in the side surface of the culture vessel 301 has been described. However, this embodiment is not limited to the exemplified configuration. One of the supply pipe 6a and the discharge pipe 6b may be arranged above the culture vessel 301 as in the first embodiment and other embodiments. Alternatively, the holes 301a and 301b may be formed in the side surface of the culture vessel 301 so as to be arranged side by side.

### Fourth Embodiment

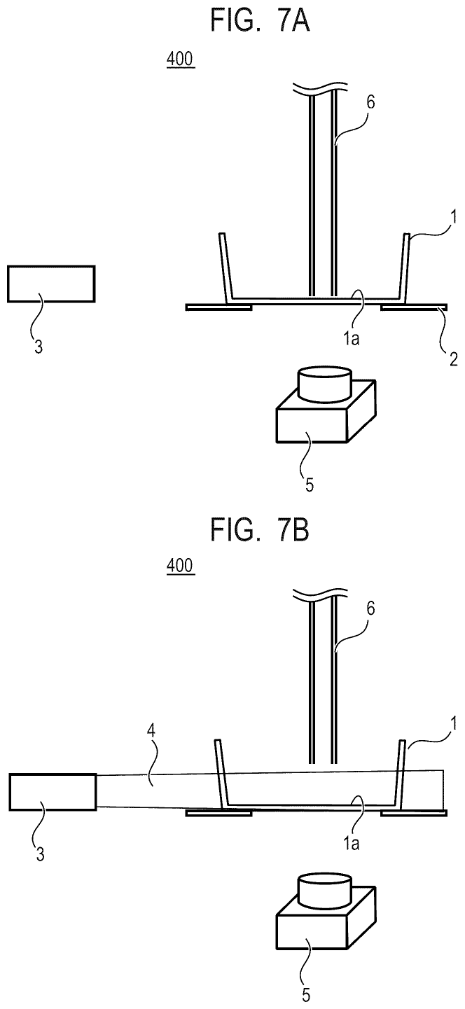

In the first and second embodiments, the pipes 6 provided to the culture vessel 1 are not moved at the time of photographing of cultured cells from their positions during cell culture, and the configurations for coping with the stray light generated in such a case have been described. Meanwhile, in this embodiment, a possibility of generation of the stray light is reduced by retreating the pipes 6 from an irradiation area with illumination light 4 at the time of photographing of cultured cells. Now, a configuration of a cell culture system 400 according to this embodiment is illustrated in FIG. 7A and FIG. 7B in the same fashion as in FIG. 1A.

In FIG. 7A, the cell culture system 400 at the time of cell culture is illustrated. A light source 3 is not emitting illumination light, and pipes 6 are arranged at positions that allow supply and discharge of water to and from cultured cells. In FIG. 7B, the cell culture system 400 at the time of photographing cells is illustrated. Illumination light 4 is emitted from the light source 3, and the pipes 6 are retreated upwards from an irradiation area by a pipe drive unit (not shown). The configuration described above prevents the generation of stray light due to the presence of the pipes 6, and a suitable cell image of a larger area can be obtained.

As described above, in the cell culture system 400 according to this embodiment, when a culture surface 1a is observed with an image pickup apparatus 5, the supply pipe 6a and the discharge pipe 6b are driven so as to be moved away from the culture vessel 1. As a result, an opening of the supply pipe 6a and an opening of the discharge pipe 6b are located outside an irradiation area with the illumination light, and the pipes themselves are also located outside the irradiation area. Thus, the stray light is not generated due to the presence of the pipes 6, and a suitable cell image of a larger area can be obtained.

The present disclosure is described above referring to the embodiments. However, the present disclosure is not limited to the above-mentioned embodiments. The present disclosure also encompasses the invention modified within a scope not deviated from the present disclosure, and the invention equivalent to the present disclosure. Further, the above-mentioned embodiments may be combined with each other as appropriate within the scope not deviated from the gist of the present invention.

According to one aspect of the present disclosure, a cell culture apparatus, a cell culture system, and a cell image pickup system, which enable automated cell culture using pipes for supplying and discharging water into and from a culture vessel can be provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-139700, filed Aug. 30, 2023, which is hereby incorporated by reference herein in its entirety.

What is claimed is:

1. A cell culture system comprising:
a cell culture apparatus including:
a placement table configured to allow placement of a culture vessel having a culture surface on which cell culture is viable and a side surface that forms a peripheral wall around the culture surface, the side surface being optically transparent;
a supply pipe configured to allow liquid to be supplied into the culture vessel; and
a discharge pipe configured to allow liquid to be discharged from the culture vessel;
an image pickup portion configured to allow observation of the culture surface; and
a light irradiation portion configured to emit illumination light toward the side surface,
wherein the supply pipe and the discharge pipe are arranged so that an opening of the supply pipe and an opening of the discharge pipe are located in proximity to points on an inner side surface of the culture vessel, which define a width of the culture surface in a direction orthogonal to an optical axis of the illumination light.

2. The cell culture system according to claim 1,
wherein, when the width of the culture surface is represented by D and two straight lines that pass through the points on the inner side of the culture vessel and are parallel to the optical axis are represented by L1 and L2, the supply pipe and the discharge pipe are arranged so as to in a plane view, make a distance between a foot of a perpendicular extended from a center of the opening of the supply pipe to the culture surface and one of the straight line L1 and the straight line L2, which is closer to the supply pipe, and a distance between a foot of a perpendicular extended from a center of the opening of the discharge pipe to the culture surface and one of the straight line L1 and the straight line L2, which is closer to the discharge pipe, each equal to D/10 or less.

3. The cell culture system according to claim 1, wherein, when the culture vessel has a rectangular shape in plan view, the supply pipe and the discharge pipe are arranged in proximity to an inner side of side surfaces of the culture vessel, the side surfaces extending in parallel to the optical axis.

4. The cell culture system according to claim 1, wherein, when the culture vessel has a rectangular shape in plan view, the supply pipe and the discharge pipe are connected to the culture vessel so as to have openings exposed on side surfaces of the culture vessel, the side surfaces extending in parallel to the optical axis.

5. The cell culture system according to claim 1, wherein, when the culture vessel has a circular shape in plan view, the supply pipe and the discharge pipe are both arranged between one of opposed surfaces of a frame having a square shape inscribed in the circle in plan view, the opposed surfaces being parallel to the optical axis, and a part of a surface defining an inner side of the circle, which is opposed to the one opposed surface, or one of the supply pipe and the discharge pipe is arranged between one of the opposed surfaces being parallel to the optical axis and a part of the surface defining the inner side of the circle, which is opposed to the one opposed surface, and another one of the supply pipe and the discharge pipe is arranged between another one of the opposed surfaces and a part of the surface defining the inner side of the circle, which is opposed to the another opposed surface.

6. The cell culture system according to claim 1, wherein, when the culture vessel has a circular shape in plan view, the supply pipe and the discharge pipe are connected to the culture vessel so that both of the supply pipe and the discharge pipe have openings exposed on a part of a surface defining an inner side of the circle, which is opposed to one of opposed surfaces of a frame having a square shape inscribed in the circle in plan view, the opposed surfaces being parallel to the optical axis, or one of the supply pipe and the discharge pipe has an opening exposed on a part of the surface defining the inner side of the circle, which is opposed to one of the opposed surfaces being parallel to the optical axis, and another one of the supply pipe and the discharge pipe has an opening exposed on a part of the surface defining the inner side of the circle, which is opposed to another one of the opposed surfaces.

7. A cell culture system comprising:
a cell culture apparatus including:
a placement table that allows placement of a culture vessel having a culture surface on which cell culture is viable and a side surface forming a peripheral wall around the culture surface, the side surface being optically transparent;
a supply pipe configured to allow liquid to be supplied into the culture vessel; and
a discharge pipe configured to allow liquid to be discharged from the culture vessel;
an image pickup portion configured to allow observation of the culture surface; and
a light irradiation portion configured to emit illumination light toward the side surface,
wherein, when the culture surface is observed with the image pickup portion, an opening of the supply pipe and an opening of the discharge pipe are located outside an irradiation area with the illumination light.

8. The cell culture system according to claim 7, wherein the opening of the supply pipe and the opening of the discharge pipe are exposed on the side surface of the culture vessel.

9. The cell culture system according to claim 7, wherein, when the culture surface is observed with the image pickup portion, the supply pipe and the discharge pipe are driven so as to be moved away from the culture vessel so that the opening of the supply pipe and the opening of the discharge pipe are located outside the irradiation area with the illumination light.

10. The cell culture system according to claim 1, wherein the illumination light is sheet-like parallel light.

11. The cell culture system according to claim 1,
wherein the culture vessel has a protrusion that is formed on a surface opposite to the culture surface and protrudes in a direction opposite to a direction toward the culture surface, and
wherein the cell culture system further comprises a configuration for shielding the protrusion from the illumination light.

12. A cell culture apparatus comprising:
a placement table that allows placement of a culture vessel having a culture surface on which cell culture is viable and a side surface forming a peripheral wall around the culture surface, the side surface being optically transparent;
a supply pipe configured to allow liquid to be supplied into the culture vessel; and
a discharge pipe configured to allow liquid to be discharged from the culture vessel,
wherein the supply pipe and the discharge pipe are arranged so that an opening of the supply pipe and an opening of the discharge pipe are located in proximity to points on an inner side surface of the culture vessel, which define a width of the culture surface in a direction orthogonal to a center axis of the culture vessel placed on the placement table in plan view.

13. A cell image pickup system comprising:
the cell culture apparatus of claim 12;
a light irradiation portion configured to be moved relative to the cell culture apparatus and be able to emit illumination light toward the side surface so that an optical axis of the illumination light matches with the center axis; and
an image pickup portion configured to allow observation of the culture surface illuminated with the illumination light in response to the illumination light.
